SEQUENCE LISTING (4 sequences)

SEQ ID NO: 1 - DNA, 1529 bp, Artificial Sequence
  Flavonifractor_SC49
tattgagagtttgatcctggctcaggatgaacgctggcggcgtgcttaacacatgcaagtcgaacggagtgctcatgacagaggattcgtccaatggagtgagttacttagtggcggacgggtgagtaacgcgtgagtaacctgccttggagtggggaataacaggtggaaacatctgctaataccgcatgatgcagttgggtcgcatggctctgactgccaaagatttatcgctctgagatggactcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcgagcgttatccggatttactgggtgtaaagggcgtgtaggcgggactgcaagtcagatgtgaaaaccatgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggtgtagagatacaccttcttcttcggaagcgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagaccgcgaggtggagcaaacccctaaaagccatcccagttcggattgcaggctgcaactcgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 2 - DNA, 1548 bp, Clostridium leptum
tttagagagtttgatcctggctcaggacgaacgctggcggcgtgcctaacacatgcaagtcgaacggagttaaattcgacacccgagtatccggccgggaggcggggtgctgggggttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatggactcgcgtccgattagccagttggcggggtaacggcccaccaaagcgacgatcggtagccggactgagaggttgaacggccacattgggactgagacacggcccagactcctacgggaggcagcagtgggggatattgcacaatgggggaaaccctgatgcagcaacgccgcgtgagggaagaaggttttcggattgtaaacctctgttcttagtgacgataatgacggtagctaaggagaaagctccggctaactacgtgccagcagccgcggtaatacgtagggagcgagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccggtgtagcggtgaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtggggggtctgaccccctccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgtctaacgaagcagagatgcattaggtgcccttcggggaaaggcgagacaggtggtgcatggttgtcgtcagctcgtgtcgtgagatgttgggttaagtcccgcaacgagcgcaacccttgtttctagttgctacgcaagagcactctagagagactgccgttgacaaaacggaggaaggtggggacgacgtcaaatcatcatgccccttatgacctgggccacacacgtactacaatggctgtaaacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggccttgtacacaccgcccgtcacaccatgggagccggtaatacccgaagccagtagttcaaccgcaaggagagcgctgtcgaaggtaggattggcgactggggtgaagtcgtaacaaggtagccgtatcggaaggtgcggctggatcacctccttt

SEQ ID NO: 3 - DNA, 1529 bp, Artificial Sequence
  Flavonifractor_SC49
tattgagagtttgatcctggctcaggatgaacgctggcggcgtgcttaacacatgcaagtcgaacggagtgctcatgacagaggattcgtccaatggagtgagttacttagtggcggacgggtgagtaacgcgtgagtaacctgccttggagtggggaataacaggtggaaacatctgctaataccgcatgatgcagttgggtcgcatggctctgactgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcgagcgttatccggatttactgggtgtaaagggcgtgtaggcgggactgcaagtcagatgtgaaaaccatgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtgggaggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggtgtagagatacaccttcttcttcggaagcgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagaccgcgaggtggagcaaacccctaaaagccatcccagttcggattgcaggctgcaactcgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 4 - DNA, 1376 bp, Artificial Sequence
  Flavonifractor_SC49
  misc_feature (543)..(543): n is a, c, g, or t
  misc_feature (955)..(956): n is a, c, g, or t
aacggagtgctcatgacagaggattcgtccaatggagtgagttacttagtggcggacgggtgagtaacgcgtgagtaacctgccttggagtggggaataacaggtggaaacatctgctaataccgcatgatgcagttgagtcgcatggctctgactgccaaagatttatcgctctgagatggactcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcgagcgttatccggatttactgggtgtaaagggcgtgtaggcgggactgcaagtcagatgtgaaaaccangggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggtgtagagatacaccttnntcttcggaagcaccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagcccgcgaggaggagcaaacccctaaaagccatcccagttcggattgcaggctgcaactcgcctgtatgaagttggaatcgctagtaatcgcggatcagcatgccgcggtgaatacgttcccgggccttgtacacaccgcccgtcacaccatgagagtcgggaacacccgaagtccgtagcctaaccgc

(19) United States
(12) Patent Application Publication
(10) Pub. No.: US 2021/0121505 A1
(43) Pub. Date: Apr. 29, 2021

(54) COMPOSITIONS AND METHODS FOR TREATING INFLAMMATORY BOWEL DISEASES

(71) Applicant: Seres Therapeutics, Inc., Cambridge, MA (US)

(72) Inventors: Madhumitha NANDAKUMAR, Arlington, MA (US); Edward J. O'BRIEN, Arlington, MA (US); Sumon DATTA, Boston, MA (US)

(73) Assignee: Seres Therapeutics, Inc., Cambridge, MA (US)

(21) Appl. No.: 17/043,545

(22) PCT Filed: Mar. 29, 2019
(86) PCT No.: PCT/US2019/025010

§ 371 Date: Sep. 29, 2020

Related U.S. Application Data
(60) Provisional application No. 62/650,002, filed on Mar. 29, 2018.

(51) Int. Cl. A61K 35/742; A61P 37/06; A61P 1/00; A61P 1/04

(52) U.S. Cl. CPC A61K 35/742; A61P 1/04; A61P 1/00; A61P 37/06

(57) ABSTRACT

Provided herein are bacterial compositions that are useful for treating and preventing complications and side effects associated with an inflammatory bowel disease.

### REFERENCE TO SEQUENCE LISTING SUBMITTED ELECTRONICALLY

The content of the electronically submitted sequence listing in ASCII text file (Name: 4268.014PC01_SequenceListing_ST25.txt; Size: 8,737 bytes; and Date of Creation: Mar. 29, 2019) filed with the application is herein incorporated by reference in its entirety.

### FIELD OF THE DISCLOSURE

The present disclosure relates to bacterial compositions that can modulate the level of bile acids when administered to a subject. Such bacterial compositions are useful for treating and/or preventing complications and side effects associated with inflammatory bowel diseases.

### BACKGROUND OF THE DISCLOSURE

Inflammatory bowel disease (IBD) is a chronic disorder of the gastrointestinal tract characterized by inflammation of the intestines or colon. Symptoms of IBD can vary but generally include abdominal cramping, persistent diarrhea, and colorectal bleeding. IBD can be debilitating and can have life-threatening complications if left untreated.

Ulcerative colitis and Crohn's disease are two main forms of IBD. Although both are disorders causing inflammation of the digestive tract, they differ as to the nature and location of the inflammatory reactions in the gastrointestinal (GI) tract. Ulcerative colitis is restricted to the colon and the anus and inflammation caused by it only affects mucosa. In contrast, Crohn's disease can affect the whole gastrointestinal tract, i.e., from mouth to anus, although it commonly affects the lower part of the small intestine (ileum).

There are no known cures for IBD. Current treatment options include drugs (e.g., anti-inflammatory agents, immunosuppressants, and antibiotics), nutrition supplements, and surgery. While such treatments can reduce the signs and symptoms of the disease, they generally have limited efficacy and/or adverse side effects. See, e.g., Martinez-Montiel M. P., et al., Clin Exp Gastroenterol 8:257-269 (2015); Cunliffe R. N., et al., Aliment Pharmacol Ther 16(4):647-662 (2002). Moreover, despite the available treatment options, IBD remains a major medical challenge, with both the incidence and prevalence of IBD increasing worldwide. M'Koma, A. E., Clin Med Insights Gastroenterol 6: 33-47 (2013). Accordingly, there is a strong need for new treatment options for IBD that are safer and more efficacious.

Bile acids, together with cholesterol, phospholipids, and bilirubin, comprise the principal constituents of bile. They are synthesized from cholesterol in the liver and secreted from hepatocytes into the bile canaliculi and subsequently stored in the gall bladder. After ingestion of food, bile flows into the duodenum, wherein it contributes to the solubilization and digestion of lipid-soluble nutrients. Thomas et al., Nat Rev Drug Discov 7(8): 678-693 (2008). Accordingly, bile acids have traditionally been described to be important for the solubilization of cholesterol in the GI tract and for stimulating the absorption of cholesterol, fat-soluble vitamins, and lipids in the intestines. Hylemon P. B., et al., J Lipid Res 50(8): 1509-1520 (2009).

More recently, bile acids have also been described as being important in many other biological processes. For instance, bile acids are now known to act, at least in part, through both G-protein-coupled receptors (GPCRs) (e.g., TGR5) and nuclear hormone receptors (e.g., farnesoid X receptor, pregnane X receptor, and vitamin D receptor) and mediate not only their own biosynthesis but also mediate the metabolism of other lipid molecules (e.g., cholesterol and triglyceride) and glucose. Thomas et al., Nat Rev Drug Discov 7(8): 678-693 (2008). Bile acids have also been described as playing a role in modulating both innate and adaptive immunity. Zhu C., et al., Clin Exp Rheumatol 34: 25-31 (2016). Therefore, biological agents that can modulate bile acid levels can be useful treatment options for IBD.

### BRIEF SUMMARY OF THE DISCLOSURE

The present disclosure is directed to a composition comprising a purified population of bacteria, wherein the purified population of bacteria comprises Flavonifractor_SC49, Clostridium leptum, or a combination thereof, and wherein the composition can modulate the level of a secondary bile acid when administered to a subject. In certain embodiments, the purified population of bacteria comprises Flavonifractor_SC49. In other embodiments, the purified population of bacteria comprises Clostridium leptum. In further embodiments, the purified population of bacteria comprises both Flavonifractor_SC49 and Clostridium leptum.

In some embodiments, the Flavonifractor_SC49 comprises a 16S rDNA sequence that is at least 95%, at least 96%, at least 97%, at least 98%, at least 99%, or 100% identical to a 16S rDNA sequence of a reference Flavonifractor_SC49 OTU (SEQ ID NOs: 1, 3, or 4).

In some embodiments, the Clostridium leptum comprises a 16S rDNA sequence that is at least 95%, at least 96%, at least 97%, at least 98%, at least 99%, or 100% identical to a 16S rDNA sequence of a reference Clostridium leptum OTU (SEQ ID NO: 2).

In some embodiments, the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or combinations thereof.

In some embodiments, the secondary bile acid comprises ursodeoxycholic acid (UDCA).

Also provided herein is a method of modulating the level of a secondary bile acid in a subject in need thereof, comprising administering to the subject an effective amount of a composition disclosed herein. The present disclosure also provides a method of ameliorating one or more signs or symptoms of an inflammatory bowel disease (IBD) or maintaining a remission of an IBD in a subject in need thereof, comprising administering to the subject an effective amount of a composition disclosed herein.

In some embodiments, the secondary bile acid comprises deoxycholic acid (DCA), 3α 12-oxo-deoxycholic acid, 3β12α-deoxycholic acid (3-isodeoxycholic acid), 7α 3-oxo-chenodeoxycholic acid, lithocholic acid (LCA), 3-oxo LCA, or combinations thereof. In certain embodiments, the administration of a composition disclosed herein increases the level of the secondary bile acid in the subject. In some embodiments, the level of the secondary bile acid is increased by at least 10%, at least 20%, at least 30%, at least 40%, at least 50%, at least 60%, at least 70%, at least 80%, or at least 90% in the subject compared to a reference (e.g., corresponding level in a subject that did not receive the composition). In some embodiments, the increase in the level of the secondary bile acid is associated with a remission of the IBD. In certain embodiments, the secondary bile acid can decrease production of TNF-α and/or increase production of IL-10 in a lipopolysaccharide (LPS)-stimulated monocyte in vitro. In further embodiments, the secondary bile acid can decrease production of TNF-α and/or increase production of IL-10 in LPS-stimulated peripheral blood mononuclear cells (PBMCs) in vitro. In some embodiments, the secondary bile acid can decrease production of IL-8 in TNFα-stimulated intestinal epithelial cells in vitro.

In some embodiments, the secondary bile acid comprises ursodeoxycholic acid (UDCA). In certain embodiments, the administration decreases the level of UDCA in the subject. In some embodiments, the level of UDCA is decreased by at least 10%, at least 20%, at least 30%, at least 40%, at least 50%, at least 60%, at least 70%, at least 80%, or at least 90% in the subject compared to a reference (e.g., corresponding level in a subject that did not receive the composition). In some embodiments, the decrease in the level of UDCA is associated with a remission of the IBD.

In some embodiments, the IBD is ulcerative colitis or Crohn's disease.

### BRIEF DESCRIPTION OF THE DRAWINGS

FIG. 1 shows a comparison of the relative concentrations (measured in μg/g of fecal sample, dry weight) of secondary bile acids associated with the 7α-dehydroxylase pathway (i.e., DCA, LCA, 3-oxo LCA, 3α 12-oxo-deoxycholic acid, and 3β 12α-deoxycholic acid) measured in fecal samples obtained from different groups of ulcerative colitis patients. The ulcerative colitis patients received one of the following regimens: (A) placebo only; (B) placebo followed by a weekly dosing of a spore population derived from the feces of a healthy human (“HHSP”); (C) vancomycin followed by weekly dosing of HHSP; and (D) vancomycin followed by daily dosing of HHSP. Bile acid concentrations were measured at four different time points: (1) baseline (i.e., prior to administration of the vancomycin or HHSP) (“Visit 1”); (2) immediately after vancomycin treatment (where required) (“Visit 4”); (3) 2 weeks after beginning the administration of the HHSP (where required) (“Visit 6”); and (4) 8 weeks after beginning the administration of the spore-forming fraction (where required) (“Visit 12”). The concentrations shown at Visit 4, Visit 6, and Visit 12 are relative to the concentrations of the total bile acids measured at baseline.

FIGS. 2A to 2G show a comparison of the concentration (measured in μg/g of fecal sample, dry weight) of different bile acids measured in fecal samples from ulcerative colitis patients who are either in remission (i.e., remitters, light gray) or not in remission (i.e., non-remitters, dark gray). The patients shown received one of the regimens described in FIG. 1. Bile acid concentrations were measured at four different time points also as described in FIG. 1. FIG. 2A shows the concentration of deoxycholic acid (DCA). FIG. 2B shows the concentration of lithocholic acid (LCA). FIG. 2C shows the concentration of 3α 12-oxo-DCA (12-oxo 3a). FIG. 2D shows the concentration of 7α 3-oxo-CDCA (3-oxo 7a). FIG. 2E shows the concentration of 3-oxo-LCA. FIG. 2F shows the concentration of 3β12α-DCA (3b 12a). FIG. 2G shows the concentration of ursodeoxycholic acid (UDCA).

FIGS. 3A to 3G show a comparison of the concentration (measured in μg/g of fecal sample, dry weight) of different bile acids measured in fecal samples from ulcerative colitis patients who received vancomycin followed by a daily dosing of a spore population derived from the feces of a healthy human (HHSP). Bile acid concentrations were measured at four different time points as described in FIG. 1. For each time point, the ulcerative colitis patients were divided into two groups: (i) in remission (i.e., remitters, light gray) or (ii) not in remission (i.e., non-remitters, dark gray). FIG. 3A shows the concentration of deoxycholic acid (DCA). FIG. 3B shows the concentration of lithocholic acid (LCA). FIG. 3C shows the concentration of 3α 12-oxo-DCA (12-oxo 3a). FIG. 3D shows the concentration of 7α 3-oxo-CDCA (3-oxo 7a). FIG. 3E shows the concentration of 3-oxo-LCA. FIG. 3F shows the concentration of 3β 12α-DCA (3b 12a). FIG. 3G shows the concentration of ursodeoxycholic acid (UDCA).

FIGS. 4A and 4B show the effect of different bile acids on the production of TNF-α (FIG. 4A) and IL-10 (FIG. 4B) in LPS-stimulated peripheral blood mononuclear cells (PBMCs) in vitro. The bile acids shown include: (i) conjugated primary bile acids (Conj. 1°)-tauro-cholic acid (tCA), glyco-cholic acid (gCA), tauro-chenodeoxycholic acid (tCDCA), and glycol-chenodeoxycholic acid (gCDCA); (ii) primary bile acids (1°)-cholic acid (CA) and chenodeoxycholic acid (CDCA); (iii) secondary bile acids (2°)-deoxycholic acid (DCA) and lithocholic acid (LCA); (iv) iso-bile acids (Iso-BA)-ursodeoxycholic acid (UDCA); and (v) conjugated secondary bile acids (Conj. 2°)-tauro-deoxycholic acid (tDCA), glyco-deoxycholic acid (gDCA), and tauro-sulfo-lithocholic acid (tLCA). For each of the bile acids, the bars shown correspond to a concentration of the bile acid used (i.e., 0, 12.5, 25, and 50 μM), with increase in concentration going from left to right. Asterisk indicates possible toxicity at the given bile acid concentration.

FIGS. 5A and 5B show the effect of different bile acids on the production of TNF-α (FIG. 5A) and IL-10 (FIG. 5B) in LPS-stimulated monocytes in vitro. The bile acids shown include: (i) conjugated primary bile acids (Conj. 1°)-tauro-cholic acid (tCA), glyco-cholic acid (gCA), tauro-chenodeoxycholic acid (tCDCA), and glycol-chenodeoxycholic acid (gCDCA); (ii) primary bile acids (1°)-cholic acid (CA) and chenodeoxycholic acid (CDCA); (iii) secondary bile acids (2°)-deoxycholic acid (DCA) and lithocholic acid (LCA); and (iv) conjugated secondary bile acids (Conj. 2°)-tauro-deoxycholic acid (tDCA), glyco-deoxycholic acid (gDCA), and tauro-sulfo-lithocholic acid (tLCA). For each of the bile acids, the bars shown correspond to a concentration of the bile acid used (i.e., 0, 12.5, 25, and 50 μM), with increase in concentration going from left to right.

FIG. 6 shows the effect of different bile acids on the production of IL-8 in TNF-α-stimulated intestinal epithelial cells (“IECs”). The bile acids shown include: (i) conjugated primary bile acids (Conj. 1°)-tauro-cholic acid (tCA), glyco-cholic acid (gCA), tauro-chenodeoxycholic acid (tCDCA), and glycol-chenodeoxycholic acid (gCDCA); (ii) primary bile acids (1°)-cholic acid (CA) and chenodeoxycholic acid (CDCA); (iii) secondary bile acids (2°)-deoxycholic acid (DCA) and lithocholic acid (LCA); (iv) iso-bile acids (Iso-BA)-ursodeoxycholic acid (UDCA); and (v) conjugated secondary bile acids (Conj. 2°)-tauro-deoxycholic acid (tDCA), glyco-deoxycholic acid (gDCA), and tauro-sulfo-lithocholic acid (tLCA). For each of the bile acids, the bars shown correspond to a concentration of the bile acid used (i.e., 62.5, 125, and 250 μM), with increase in concentration going from left to right. The amount of IL-8 produced in the presence of different bile acids is shown relative to the no bile acid control. Asterisk indicates possible toxicity at the given bile acid concentration.

FIG. 7 provides a comparison of the total amount of certain secondary bile acids (i.e., those associated with the 7α-dehydroxylase pathway—DCA, LCA, 3-oxo LCA, 3α 12-oxo-deoxycholic acid, and 3β 12α-deoxycholic acid) measured in fecal samples from different ulcerative colitis patients to the presence or absence of species comprising Flavonifractor_SC49 and/or Clostridium leptum. Prior to the measurement of the secondary bile acids, the ulcerative colitis patients received one of the regimens described in FIG. 1. The patient samples were divided based on the presence of Flavonifractor_SC49 and Clostridium leptum: (i) none (“1”); (ii) Flavonifractor_SC49 only (“2”); (iii) Clostridium leptum only (“3”); or (iv) both (“4”).

### DETAILED DESCRIPTION OF THE DISCLOSURE

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, 5 percent, 3 percent, 2 percent, or 1 percent; up or down (higher or lower).

The term “bile acids” refers to a family of molecules, composed of a steroid structure with four rings, a five or eight carbon side-chain terminating in a carboxylic acid joined at the 17-position of the steroid scaffold, and the presence and orientation of different numbers of hydroxy groups. Depending on the tissue, the structure of the bile acids can vary. For instance, upon their synthesis in the liver, the bile acids are conjugated to either taurine or glycine residues (“conjugated primary bile acids”) and subsequently excreted and stored in the gall bladder. During digestion, the conjugated primary bile acids are then secreted into the intestinal lumen. In some embodiments, the primary conjugated bile acids are glycocholic acid (gCA), taurocholic acid (tCA), glycochenodeoxycholic acid (gCDCA), or taurochenodeoxycholic acid (tCDCA).

Within the intestinal lumen, the resident intestinal bacteria express enzymes (e.g., bile salt hydrolase (BSH)), which deconjugate the conjugated primary bile acids to produce “primary bile acids.” In some embodiments, the primary bile acids comprise cholic acid (CA) or chenodeoxycholic acid (CDCA). Primary bile acids are then further processed (via enzymes, such as hydroxysteroid dehydrogenase (HSDH) or 7α-dehydrogenase) to become “secondary bile acids.” In some embodiments, the secondary bile acids comprise deoxycholic acid (DCA), oxo-deoxycholic acid (3 or 12), iso-deoxycholic acid (3 or 12), oxo-cholic acid (3, 7, or 12), iso-cholic acid (3, 7, or 12), lithocholic acid (LCA), oxo-LCA, iso-LCA, oxo-chenodeoxy cholic acid (3 or 7), or iso-chenodeoxy cholic acid (3 or 7).

The secondary bile acids produced in the intestinal lumen can circulate back to the liver, where they are reconjugated to become “conjugated secondary bile acids.” In some embodiments, the secondary conjugated bile acids of the present disclosure comprise glyco-iso-deoxycholic acid (3 or 12), tauro-iso-deoxycholic acid (3 or 12), glyco-deoxycholic acid, tauro-deoxycholic acid, glyco-iso-cholic acid (3, 7, or 12), tauro-iso-cholic acid (3, 7, or 12), sulfo-lithocholic acid, glyco-sulfo-lithocholic acid, tauro-sulfo-lithocholic acid, glyco-iso-chenodeoxycholic acid (3 or 7), tauro-iso-chenodeoxycholic acid (3 or 7), glyco-oxo-chenodeoxycholic acid (3 or 7), or tauro-oxo-chenodeoxycholic acid (3 or 7).

The term “clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

The term “microbiota” refers to the ecological community of microorganisms that occur (sustainably or transiently) in and on an animal subject, typically a mammal such as a human, including eukaryotes, archaea, bacteria, and viruses (including bacterial viruses, i.e., phage).

The term “microbiome” refers to the microbes that live in and on the human body, both sustainably and transiently, including eukaryotes, archaea, bacteria, and viruses (including bacterial viruses (i.e., phage)). As used herein, “genetic content” includes genomic DNA, RNA such as ribosomal RNA, the epigenome, plasmids, and all other types of genetic information.

As used herein, the term “microbial augmentation” or simply “augmentation” refers to the establishment or significant increase of a population of microbes that are (i) absent or undetectable (as determined by the use of standard genomic and microbiological techniques) from the administered therapeutic microbial composition, (ii) absent, undetectable, or present at low frequencies in the host niche (as example: gastrointestinal tract, skin, anterior-nares, or vagina) before the delivery of the microbial composition, and (iii) are found after the administration of the microbial composition or significantly increase, for instance 2-fold, 5-fold, 1×10², 1×10³, 1×10⁴, 1×10⁵, 1×10⁶, 1×10⁷, or greater than 1×10⁸, in cases where they were present at low frequencies. The microbes that comprise an augmented ecology can be derived from exogenous sources such as food and the environment, or grow out from micro-niches within the host where they reside at low frequency.

Without committing to any theory, the administration of the therapeutic microbial composition may induce an environmental shift in the target niche that promotes favorable conditions for the growth of certain commensal microbes. In the absence of treatment with a therapeutic microbial composition, although the host may be exposed to these microbes, sustained growth and the positive health effects associated with the stable population of increased levels of the microbes comprising the augmented ecology are not observed or are less frequently observed in a targeted population.

The term “microbial engraftment” or “engraftment” refers to the establishment of OTUs comprising a therapeutic microbial composition, e.g., a bacterial composition, in a target niche that are absent or undetectable in the treated host prior to treatment. The microbes that comprise the engrafted ecology are present in the therapeutic microbial composition and establish as constituents of the host microbial ecology upon treatment. Engrafted OTUs can establish for a transient period of time, or demonstrate long-term stability in the microbial ecology that populates the host post treatment with a therapeutic microbial composition. Without committing to any theory, the engrafted ecology may induce an environmental shift in the target niche that promotes favorable conditions for the growth of commensal microbes capable of catalyzing a shift from a dysbiotic ecology to one representative of a health state.

The term “ecological niche” or “niche” refers to the ecological space in which an organism or group of organisms occupies. Niche describes how an organism or population or organisms responds to the distribution of resources, physical parameters (e.g., host tissue space) and competitors (e.g., by growing when resources are abundant, and when predators, parasites and pathogens are scarce) and how it in turn alters those same factors (e.g., limiting access to resources by other organisms, acting as a food source for predators and a consumer of prey).

The term “dysbiosis” refers to a state of the microbiota of the GI tract or other body area in a subject, including mucosal or skin surfaces in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

As used herein, the term “operational taxonomic units” or “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence can be the 16S rDNA sequence or a portion of the 16S rDNA sequence. In other embodiments, the entire genomes of two entities are sequenced and compared. In another embodiment, select regions such as multilocus sequence tags (MLST), specific genes, or sets of genes can be genetically compared. In 16S embodiments, OTUs that share ≥97% average nucleotide identity across the entire 16S or a variable region of the 16S rDNA, e.g., a V4 region, are considered the same OTU (see, e.g., Claesson M J, Wang Q, O'Sullivan O, Greene-Diniz R, Cole J R, Ros R P, and O'Toole P W. 2010. Comparison of two next-generation sequencing technologies for resolving highly complex microbiome composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940). In embodiments involving the complete genome, MLSTs, specific genes, or sets of genes OTUs that share ≥95% average nucleotide identity are considered the same OTU (see, e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940.). OTUs are frequently defined by comparing sequences between organisms. Generally, sequences with less than 95% sequence identity are not considered to form part of the same OTU. OTUs can also be characterized by any combination of nucleotide markers or genes, in particular highly conserved genes (e.g., “house-keeping” genes), or a combination thereof. Such characterization employs, e.g., WGS data or a whole genome sequence.

As used herein, the term “phylogenetic tree” refers to a graphical representation of the evolutionary relationships of one genetic sequence to another that is generated using a defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

“Residual habitat products” refers to material derived from the habitat of a microbiota within or on a human or animal excluding the microbiota. An individual's microbiota are in, for example, feces in the gastrointestinal tract, on the skin itself, in saliva, mucus of the respiratory tract, or secretions of the genitourinary tract, all of which contain biological and other matter associated with the microbial community. “Substantially free of residual habitat products” means that the bacterial composition contains a reduced amount of the biological matter associated with the microbial environment on or in the human or animal subject and is 100% free, 99% free, 98% free, 97% free, 96% free, or 95% free of any contaminating biological matter associated with the microbial community or the contaminating matter is below a level of detection. Residual habitat products can include abiotic materials (including undigested food) or it can include unwanted microorganisms. Substantially free of residual habitat products can also mean that the bacterial composition contains no detectable cells from a human or animal and that only microbial cells are detectable. In some embodiments, substantially free of residual habitat products can mean that the bacterial composition contains no detectable viral (including bacterial viruses (i.e., phage)), fungal, mycoplasmal contaminants. In other embodiments, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the bacterial composition are human or animal, as compared to microbial cells. There are multiple ways to accomplish reduced presence of residual habitat products, none of which are limiting. Thus, contamination can be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate reduction of residual habitat products include genetic analysis (e.g., PCR, DNA sequencing), serology and antigen analysis, enzymatic and metabolic analysis, and methods using instrumentation such as flow cytometry with reagents that distinguish desired constituents from contaminants.

The term “16S sequencing” or “16S rDNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria.

The term “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75(10):4801-4805 (1978). In some embodiments, at least one of the V1, V2, V3, V4, V5, V6, V7, V8, and V9 regions are used to characterize an OTU. In some embodiments, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

As used herein, the term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject can be suffering from a dysbiosis, including, but not limited to, an infection due to a gastrointestinal pathogen or can be at risk of developing or transmitting to others an infection due to a gastrointestinal pathogen. In some embodiments, the subject is suffering from an inflammatory bowel disease (e.g., ulcerative colitis or Crohn's disease).

As used herein, “a subject having inflammatory bowel disease” is synonymous with the term “a subject diagnosed with having an inflammatory bowel disease,” and means a patient having Crohn's disease or ulcerative colitis. Crohn's disease (regional enteritis) is a disease of chronic inflammation that can involve any part of the gastrointestinal tract. Commonly, the distal portion of the small intestine (ileum) and cecum are affected. In other cases, the disease is confined to the small intestine, colon or anorectal region. Crohn's disease occasionally involves the duodenum and stomach, and more rarely the esophagus and oral cavity. Useful compositions as described herein can ameliorate or prevent one of more signs or symptoms of an IBD, non-limiting examples of which are described herein.

The variable clinical manifestations of Crohn's disease are, in part, a result of the varying anatomic localization of the disease. The most frequent symptoms of CD are abdominal pain, diarrhea and recurrent fever. CD is commonly associated with intestinal obstruction or fistula, which is an abnormal passage between diseased loops of bowel, for example. Crohn's disease also includes complications such as inflammation of the eye, joints and skin; liver disease; kidney stones or amyloidosis. In addition, CD is associated with an increased risk of intestinal cancer.

Several features are characteristic of the pathology of Crohn's disease. The inflammation associated with CD, known as transmural inflammation, involves all layers of the bowel wall. Thickening and edema, for example, typically also appear throughout the bowel wall, with fibrosis also present in long-standing disease. The inflammation characteristic of CD also is discontinuous in that segments of inflamed tissue, known as “skip lesions,” are separated by apparently normal intestine. Furthermore, linear ulcerations, edema, and inflammation of the intervening tissue lead to a “cobblestone” appearance of the intestinal mucosa, which is distinctive of CD.

A hallmark of Crohn's disease is the presence of discrete aggregations of inflammatory cells, known as granulomas, which are generally found in the submucosa. Some Crohn's disease cases display the typical discrete granulomas, while others show nonspecific transmural inflammation. As a result, the presence of discrete granulomas is indicative of CD, although the absence of granulomas also is consistent with the disease. Thus, transmural or discontinuous inflammation, rather than the presence of granulomas, is a preferred diagnostic indicator of Crohn's disease (Rubin and Farber, Pathology (Second Edition) Philadelphia: J. B. Lippincott Company (1994)).

Ulcerative colitis (UC) is a disease of the large intestine characterized by chronic diarrhea with cramping abdominal pain, rectal bleeding, and loose discharges of blood, pus and mucus. The manifestations of ulcerative colitis vary widely. A pattern of exacerbations and remissions typifies the clinical course of most UC patients (70%), although continuous symptoms without remission are present in some patients with UC. Local and systemic complications of UC include arthritis, eye inflammation such as uveitis, skin ulcers and liver disease. In addition, ulcerative colitis and especially long-standing, extensive disease is associated with an increased risk of colon carcinoma.

Several pathologic features characterize UC in distinction to other inflammatory bowel diseases. Ulcerative colitis is a diffuse disease that usually extends from the most distal part of the rectum for a variable distance proximally. The term left-sided colitis describes an inflammation that involves the distal portion of the colon, extending as far as the splenic flexure. Sparing of the rectum or involvement of the right side (proximal portion) of the colon alone is unusual in ulcerative colitis. The inflammatory process of ulcerative colitis is limited to the colon and does not involve, for example, the small intestine, stomach or esophagus. In addition, ulcerative colitis is distinguished by a superficial inflammation of the mucosa that generally spares the deeper layers of the bowel wall. Crypt abscesses, in which degenerated intestinal crypts are filled with neutrophils, also are typical of ulcerative colitis (Rubin and Farber, supra, 1994).

In comparison with Crohn's disease, which is a patchy disease with frequent sparing of the rectum, ulcerative colitis is characterized by a continuous inflammation of the colon that usually is more severe distally than proximally. The inflammation in ulcerative colitis is superficial in that it is usually limited to the mucosal layer and is characterized by an acute inflammatory infiltrate with neutrophils and crypt abscesses. In contrast, Crohn's disease affects the entire thickness of the bowel wall with granulomas often, although not always, present. Disease that terminates at the ileocecal valve, or in the colon distal to it, is indicative of ulcerative colitis, while involvement of the terminal ileum, a cobblestone-like appearance, discrete ulcers or fistulas suggest Crohn's disease.

In addition to IBD, the bacterial compositions disclosed herein can also be useful for the treatment of other immune-mediated gastrointestinal disorders, including, but not limited to, lymphocytic colitis; microscopic colitis; collagenous colitis; autoimmune enteropathy, including autoimmune enteritis and autoimmune enterocolitis; allergic gastrointestinal disease; and eosinophilic gastrointestinal disease, including eosinophilic gastroenteritis and eosinophilic enteropathy.

As used herein, the term “spore” or “endospore” refers to an entity, particularly a bacterial entity, which is in a dormant, non-vegetative and non-reproductive stage. Spores are generally resistant to environmental stress such as radiation, desiccation, enzymatic treatment, temperature variation, nutrient deprivation, and chemical disinfectants. In some embodiments, a spore or spore population is resistant to 50% ethanol.

A “spore population” refers to a plurality of spores present in a composition. Synonymous terms used herein include spore composition, spore preparation, ethanol treated spore fraction and spore ecology. A spore population can be purified from a fecal donation, e.g., via ethanol or heat treatment, or a density gradient separation or any combination of methods described herein to increase the purity, potency and/or concentration of spores in a sample. Alternatively, a spore population can be derived through culture methods starting from isolated spore former species or spore former OTUs or from a mixture of such species, either in vegetative or spore form.

In some embodiments, the spore preparation comprises spore forming species wherein residual non-spore forming species have been inactivated by chemical or physical treatments including ethanol, detergent, heat, sonication, and the like; or wherein the non-spore forming species have been removed from the spore preparation by various separations steps including density gradients, centrifugation, filtration and/or chromatography; or wherein inactivation and separation methods are combined to make the spore preparation. In yet another embodiment, the spore preparation comprises spore forming species that are enriched over viable non-spore formers or vegetative forms of spore formers. In this embodiment, spores are enriched by 2-fold, 5-fold, 10-fold, 50-fold, 100-fold, 1000-fold, 10,000-fold or greater than 10,000-fold compared to all vegetative forms of bacteria. In yet another embodiment, the spores in the spore preparation undergo partial germination during processing and formulation such that the final composition comprises spores and vegetative bacteria derived from spore forming species.

The term “germinant” refers to a material or composition or physical-chemical process capable of inducing vegetative growth of a bacterium that is in a dormant spore form, or group of bacteria in the spore form, either directly or indirectly in a host organism and/or in vitro.

The term “sporulation induction agent” refers to a material or physical-chemical process that is capable of inducing sporulation in a bacterium, either directly or indirectly, in a host organism and/or in vitro.

The term “increase production of bacterial spores” includes an activity or a sporulation induction agent. “Production” in this context includes conversion of vegetative bacterial cells into spores and augmentation of the rate of such conversion, as well as decreasing the germination of bacteria in spore form, decreasing the rate of spore decay in vivo, or ex vivo, or to increasing the total output of spores (e.g., via an increase in volumetric output of fecal material).

The “colonization” of a host organism includes the non-transitory residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens can be demonstrated, e.g., by a biopsy sample, or reductions can be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject. Non-comestible products are often found in preparations of bacteria from the prior art.

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the) XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov. Other methods of determining identity that are known in the art can be used.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “μg” and “μM,” respectively.

Various aspects described herein are described in further detail in the following subsections.

### I. Bacterial Compositions

Described herein are bacteria and combinations of bacteria of the human gastrointestinal tract microbiota with the capacity to modulate the level of one or more bile acids in a subject. In some embodiments, the modulation of the bile acid level can treat, prevent, delay, or ameliorate one or more signs or symptoms associated with a gastrointestinal disorder, e.g., an inflammatory bowel disease (e.g., ulcerative colitis or Crohn's disease). Without being limited to a specific mechanism, it is thought that the increase and/or decrease of certain bile acids can reduce the amount of pro-inflammatory mediators (e.g., TNF-α or IL-8) produced by activated cells (e.g., monocytes, PBMCs, or intestinal epithelial cells). In some embodiments, the increase and/or decrease of certain bile acids can increase the amount of anti-inflammatory mediators (e.g., IL-10) produced by the activated cells.

In some embodiments, a bacterial composition disclosed herein comprises two types of bacteria (termed “binary combinations” or “binary pairs”). In some embodiments, a bacterial composition comprises more than two types of bacteria. Accordingly, in some embodiments, a bacterial composition of the present disclosure comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50, or greater than 50 types of bacteria, as defined by species or operational taxonomic unit (OTU), or otherwise as provided herein.

In some embodiments, the types of bacteria found in a bacterial composition disclosed herein are those that are found to be depleted in IBD patients and/or are typically present only in low levels or is absent in patients diagnosed with an IBD (e.g., patients with active disease). In some embodiments, a bacterial composition includes one or more additional bacteria that are present with high frequency in a population of healthy humans.

In some embodiments, the first and/or the second type of bacteria comprises a 16S rDNA sequence that is at least 85%, at least 90%, at least 95%, at least 96%, at least 97%, at least 98%, at least 99%, or 100% identical to a 16S rDNA sequence of a reference Flavonifractor_SC49 OTU (SEQ ID NO: 1, 3, or 4). Flavonifractor_SC49 disclosed herein is a novel species that belongs to the genus of the family Ruminococcaceae. In some embodiments, the first and/or the second type of bacteria comprises a 16S rDNA sequence that is at least 85%, at least 90%, at least 95%, at least 96%, at least 97%, at least 98%, at least 99%, or 100% identical to a 16S rDNA sequence of a reference Clostridium leptum OTU (SEQ ID NO: 2). In some embodiments, the first type of bacteria comprises a 16S rDNA sequence that is at least 85%, at least 90%, at least 95%, at least 96%, at least 97%, at least 98%, at least 99%, or 100% identical to SEQ ID NO: 1, 3, or 4, and the second type of bacteria comprises a 16S rDNA sequence that is at least 85%, at least 90%, at least 95%, at least 96%, at least 97%, at least 98%, at least 99%, or 100% identical to SEQ ID NO: 2. In some embodiments, the bacteria Flavonifractor_SC49 and/or Clostridium leptum are more prevalent in IBD patients who are in remission (i.e., no active flare-up of disease) compared to IBD patients with active disease.

In some embodiments, the strain of an OTU useful for the present disclosure (e.g., an OTU disclosed herein) can be obtained from a public biological resource center such as the ATCC (atcc.org), the DSMZ (dsmz.de), or the Riken BioResource Center (en.brc.riken.jp). Methods for determining sequence identity are known in the art.

In some embodiments, a bacterial composition can modulate the level of a bile acid in a subject. In some embodiments, a bacterial composition increases the level of a secondary bile acid, wherein the secondary bile acid is selected from the group consisting of deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, oxo-LCA, iso-LCA, and combinations thereof. In some embodiments, the level of the secondary bile acid is increased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to a corresponding level in a reference sample. In some embodiments, the reference sample is a biological sample (e.g., fecal sample) obtained from the subject prior to the administration of a bacterial composition disclosed herein. In other embodiments, the reference sample is a biological sample (e.g., fecal sample) obtained from a subject with an active flare-up of an inflammatory bowel disease (e.g., ulcerative colitis or Crohn's disease).

In some embodiments, the increase in the level of a secondary bile acid reduces the amount of pro-inflammatory mediators (e.g., TNF-α or IL-8) produced by activated cells (e.g., LPS-stimulated monocytes, LPS-stimulated PBMCs, or TNF-α-stimulated intestinal epithelial cells). In some embodiments, the increase in the level of a secondary bile acid increases the amount of anti-inflammatory mediators (e.g., IL-10) produced by the activated cells. In certain embodiments, the amount of pro-inflammatory mediators produced is decreased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to a reference sample (e.g., activated cells not treated with increased concentration of a secondary bile acid). In some embodiments, the amount of anti-inflammatory mediators produced is increased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to a reference sample (e.g., activated cells not treated with increased concentration of a secondary bile acid). Accordingly, in some embodiments, a bacterial composition of the present disclosure has an anti-inflammatory effect when administered to a subject.

In some embodiments, a bacterial composition can also decrease the level of ursodeoxycholic acid (UDCA). In some embodiments, the level of UDCA is decreased by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to a corresponding level in a reference sample. In certain embodiments, the reference sample is a biological sample (e.g., fecal sample) obtained from the subject prior to the administration of a bacterial composition disclosed herein. In other embodiments, the reference sample is a biological sample (e.g., fecal sample) obtained from a subject with an active flare-up of an inflammatory bowel disease (e.g., ulcerative colitis or Crohn's disease). In some embodiments, UDCA can increase the amount of a pro-inflammatory mediator (e.g., IL-8) produced by, e.g., intestinal epithelial cells. Accordingly, in some embodiments, the decrease in the level of UDCA can reduce the amount of one or more pro-inflammatory mediators (e.g., IL-8) produced by, e.g., intestinal epithelial cells.

In some embodiments, a bacterial composition can modulate the level of a bile acid in a subject by altering the activity of one or more enzymes involved in bile acid synthesis. In some embodiments, a bacterial composition can modulate the activity of bile salt hydrolase (BSH), 7α-dehydroxylase, and/or hydroxysteroid dehydrogenase (HSDH).

In some embodiments, a bacterial composition of the present disclosure comprises bacteria that are capable of forming spores (i.e., spore-forming bacteria). Accordingly, in some embodiments, a bacterial composition comprises a purified population of bacteria, wherein the bacteria are in the form of spores. In some embodiments, all the bacteria are in the form of spores. In other embodiments, some of the bacteria are in the form of spores, while other bacteria are not in the form of spores (i.e., vegetative-state). In some embodiments, the bacterial composition comprises a purified population of spore-forming bacteria, wherein the bacteria are all in the vegetative-state.

As used herein, the terms “purify”, “purified” and “purifying” refer to the state of a population (e.g., a plurality of known or unknown amount and/or concentration) of desired bacteria or bacterial spores, that have undergone one or more processes of purification, e.g., a selection or an enrichment of the desired bacterium and/or bacterial spore, or alternatively a removal or reduction of residual habitat products as described herein. In some embodiments, a purified population has no detectable undesired activity or, alternatively, the level or amount of the undesired activity is at or below an acceptable level or amount. In other embodiments, a purified population has an amount and/or concentration of desired bacteria or bacterial spores, e.g., in general or of selected species, at or above an acceptable amount and/or concentration. In other embodiments, the ratio of desired-to-undesired activity (e.g., spores compared to vegetative bacteria), has changed by 2-, 5-, 10-, 30-, 100-, 300-, 1×10⁴, 1×10⁵, 1×10⁶, 1×10⁷, 1×10⁸, or greater than 1×10⁸. In other embodiments, a purified population of bacterial spores is enriched as compared to the starting material (e.g., a fecal material) from which the population is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, 99.9999%, or greater than 99.999999% as compared to the starting material.

In some embodiments, a purified population of bacteria has reduced or undetectable levels of one or more pathogenic activities, such as toxicity, an ability to cause infection of the mammalian recipient subject, an undesired immunomodulatory activity, an autoimmune response, a metabolic response, or an inflammatory response or a neurological response. In some embodiments, the pathogenic activity of the bacteria is reduced by at least 5%, at least 10%, at least 20%, at least 30%, at least 40%, at least 50%, at least 60%, at least 70%, at least 80%, at least 90%, at least 95%, at least 96%, at least 97%, at least 98%, at least 99% compared to the reference bacteria. In some embodiments, a purified population of bacteria has reduced sensory components as compared to fecal matter, such as reduced odor, taste, appearance, and umami.

In some embodiments, a bacterial composition disclosed herein is substantially free of residual habitat products and/or substantially free of a detectable level of a pathogenic material (e.g., contains no detectable viral (including bacterial viruses (i.e., phage)), fungal, mycoplasmal, or toxoplasmal contaminants, or eukaryotic parasites, such as a helminth. In some embodiments, a bacterial composition is substantially free of acellular material (e.g., DNA, viral coat material, or non-viable bacterial material).

In some embodiments, a bacterial composition comprises a population of bacteria that are sensitive to one or more antibiotics that can be used in a human. In some embodiments, bacteria of the composition are resistant to one or more antibiotics that are used to prophylactically treat patients with an active IBD (e.g., acute flare-up). Such antibiotics include, but are not limited to, β-lactams, vancomycin, aminoglycosides, fluoroquinolones, and daptomycin.

In some embodiments, a bacterial composition comprises a population of bacteria that have been purified from a biological material (e.g., fecal materials, such as feces or materials isolated from the various segments of the small and large intestines) obtained from a mammalian donor subject. In some embodiments, the biological material (e.g., fecal material) can be obtained from multiple donors (e.g., 2, 3, 4, 5, 6, 7, 8, 9, 10, 15, 20, 25, 30, 35, 40, 45, 50, 75, 100, 200, 300, 400, 500, 750, 1000 or from greater than 1000 donors), where such materials are then pooled prior to purification of the desired bacteria. In other embodiments, the biological material can be obtained from a single donor subject over multiple times and pooled from multiple samples, e.g., 2, 3, 4, 5, 6, 7, 8, 9, 10, 15, 20, 25, 30, 32, 35, 40, 45, 48, 50, 100 samples from a single donor.

Mammalian donor subjects useful for the present disclosure are generally of good health and have microbiota consistent with such good health. In general, a donor subject has not been administered antibiotic compounds within a specified period prior to the collection of the fecal material. In certain embodiments, a donor subject is not obese or overweight, and can have body mass index (BMI) scores of below 25, such as between 18.5 and 24.9. In other embodiments, a donor subject is not mentally ill or has no history or familial history of mental illness, such as anxiety disorder, depression, bipolar disorder, autism spectrum disorders, schizophrenia, panic disorders, attention deficit (hyperactivity) disorders, eating disorders or mood disorders. In other embodiments, a donor subject does not have irritable bowel disease (e.g., Crohn's disease or ulcerative colitis), irritable bowel syndrome, celiac disease, colorectal cancer or a family history of these diseases. In other embodiments, a donor subject has been screened for blood borne pathogens and fecal transmissible pathogens using standard techniques known to one in the art (e.g., nucleic acid testing, serological testing, antigen testing, culturing techniques, enzymatic assays, assays of cell free fecal filtrates looking for toxins on susceptible cell culture substrates).

In some embodiments, a donor is also selected for the presence of certain genera and/or species that provide increased efficacy of therapeutic compositions containing these genera or species. In certain embodiments, a donor is selected for the presence of Flavornifactor_SC49 and/or Clostridium leptum. In some embodiments, a donor is selected for the presence of bacteria comprising a 16S rDNA sequence that is at least at least 85%, at least 90%, at least 95%, at least 96%, at least 97%, at least 98%, at least 99%, or 100% identical to a 16S rDNA sequence of a reference Flavonifractor_SC49 OTU (SEQ ID NO: 1, 3, or 4) or a reference Clostridium leptum OTU (SEQ ID NO: 2). In other embodiments, a donor is selected that produce relatively higher concentrations of spores in fecal material than other donors. In further embodiments, a donor is selected that provide fecal material from which spores having increased efficacy are purified; this increased efficacy is measured using in vitro or in animal studies as described below. In some embodiments, a donor can be subjected to one or more pre-donation treatments in order to reduce undesired material in the fecal material, and/or increase desired spore populations.

It is advantageous to screen the health of a donor subject prior to and optionally, one or more times after, the collection of the fecal material. Such screening identifies donors carrying pathogenic materials such as viruses (HIV, hepatitis, polio) and pathogenic bacteria. Post-collection, donors are screened about one week, two weeks, three weeks, one month, two months, three months, six months, one year or more than one year, and the frequency of such screening can be daily, weekly, bi-weekly, monthly, bi-monthly, semi-yearly or yearly. Donors that are screened and do not test positive, either before or after donation or both, are considered “validated” donors.

Methods of purifying bacteria from the biological sample (e.g., fecal sample) from donor subjects are known in the art, as described in, e.g., U.S. Pat. No. 9,011,834, which is incorporated herein by reference in its entirety.

### II. Formulations

Further provided herein are formulations for administration to humans and other subjects in need thereof (e.g., an IBD patients, e.g., an ulcerative colitis patient). Generally, a bacterial composition as described herein is combined with additional active and/or inactive materials to produce a final product, which can be in single dosage unit or in a multi-dose format.

In some embodiments, a bacterial composition comprises at least one carbohydrate. A “carbohydrate” refers to a sugar or polymer of sugars. The terms “saccharide,” “polysaccharide,” “carbohydrate,” and “oligosaccharide” can be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers.

In some embodiments, a bacterial composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments, a bacterial composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, a bacterial composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, a bacterial composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, a bacterial composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, a bacterial composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, a bacterial composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, a bacterial composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, a bacterial composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

The weight fraction of the excipient or combination of excipients in the formulation is usually about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The bacterial compositions disclosed herein can be formulated into a variety of forms and administered by a number of different means. A bacterial composition can be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. In an exemplary embodiment, the bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as taste-masking agents.

Tablets, pills, and the like can be compressed, multiply compressed, multiply layered, and/or coated. The coating can be single or multiple. In some embodiments, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In some embodiments the coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying a bacterial composition disclosed herein can be incorporated into a food product. In some embodiments the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some embodiments, a bacterial composition disclosed herein is incorporated into a therapeutic food. In some embodiments, the therapeutic food is a ready-to-use food that optionally contains some or all essential macronutrients and micronutrients. In some embodiments, a bacterial composition disclosed herein is incorporated into a supplementary food that is designed to be blended into an existing meal. In some embodiments, the supplemental food contains some or all essential macronutrients and micronutrients. In some embodiments, a bacterial composition disclosed herein is blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

In some embodiments, the formulations are filled into gelatin capsules for oral administration. An example of an appropriate capsule is a 250 mg gelatin capsule containing from 10 (up to 100 mg) of lyophilized powder (10⁸ to 10¹¹ bacteria), 160 mg microcrystalline cellulose, 77.5 mg gelatin, and 2.5 mg magnesium stearate. In other embodiments, from 10⁵ to 10¹² bacteria can be used, 10⁵ to 10⁷, 10⁶ to 10⁷, or 10⁸ to 10¹⁰, with attendant adjustments of the excipients if necessary. In further embodiments, an enteric-coated capsule or tablet or with a buffering or protective composition can be used.

In some embodiments, the number of bacteria of each type can be present in the same amount or in different amounts. For example, in a bacterial composition with two types of bacteria, the bacteria can be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three types of bacteria, the ratio of type of bacteria can be chosen pairwise from ratios for bacterial compositions with two types of bacteria. For example, in a bacterial composition comprising bacteria A, B, and C, at least one of the ratio between bacteria A and B, the ratio between bacteria B and C, and the ratio between bacteria A and C can be chosen, independently, from the pairwise combinations above.

### III. Methods of Treating a Subject

The formulations disclosed herein can be used for the treatment of an inflammatory bowel disease (IBD) (e.g., ulcerative colitis or Crohn's disease), e.g., by ameliorating one or more sign or symptom of the disease (e.g., induce remission), and/or to reduce the recurrence of active disease (e.g., maintain remission).

In some embodiments, treatment with a formulation disclosed herein increases the level of a secondary bile acid, wherein the secondary bile acid is selected from the group consisting of deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, oxo-LCA, iso-LCA, and combinations thereof. In some embodiments, treatment with a formulation of the present disclosure decreases the level of a secondary bile acid, wherein the secondary bile acid is UDCA. In some embodiments, UDCA can increase the amount of pro-inflammatory mediators (e.g., IL-8) produced by, e.g., intestinal epithelial cells.

In some embodiments, the increase and/or decrease in certain secondary bile acids is associated with at least one of the following: (i) an increase in the diversity of the gastrointestinal (GI) microbiome in a patient diagnosed with an IBD (e.g., ulcerative colitis or Crohn's disease), (ii) a reduction in GI inflammation in a patient (e.g., diagnosed with an IBD), (iii) improvement in mucosal and epithelial barrier integrity in a population of IBD patients compared to untreated IBD patients, (iv) improvement in mucosal and epithelial barrier integrity in an IBD patient compared to before treatment, (v) promotion of mucosal healing (which can be assessed, for example, by a reduction in endoscopic Mayo scores), and (vi) other improvements of at least one sign or symptom of an IBD (e.g., disease remission). Such improvements can also include, for example, improvements detected via biomarkers, such as a decrease in fecal calprotectin following treatment. Mayo scores are known in the art, e.g., see globalrph.com/mayo_clinic_score.htm. A reduction in total Mayo score from a pre-treatment score and/or improvements in rectal bleeding and/or endoscopic subscores are indicative of a therapeutic effect.

In some embodiments, the clinical remission rate after treatment with a formulation described herein is at least 20%, 25%, 30%, 40%, 50%, 60%, 70%, 75%, 80%, 90%, 95%, or 100%. In some embodiments, the clinical remission rate is improved compared to placebo, e.g., at least 60% versus 30%, respectively. In some embodiments, clinical remission is a Mayo score of points, no individual subscore >1. In some embodiments, the clinical response to treatment with a formulation is improved versus placebo, e.g., at least 60% compared to 30%, respectively. Mucosal healing is defined as a 0 or 1 on the endoscopy subscore of the Mayo score. A clinical response is, in some embodiments, a decrease from baseline in the Mayo score by AO % and A points, accompanied by a decrease in the rectal bleeding subscore of or a rectal bleeding subscore of 0 or 1. In some embodiments, clinical response is defined as a decrease of ≥3 points in Total Modified Mayo Score (TMMS) from baseline, along with at least one of a decrease of >1 point in rectal bleeding subscore or absolute rectal bleeding subscore of 0 or 1. Complete remission is defined as a TMMS <2 and an endoscopic subscore of 0 with no erythema, no blood, and no evidence of inflammation. Endoscopic improvement is defined as a decrease in the modified Mayo endoscopic subscore >1.

The formulations disclosed herein can be useful in a variety of clinical situations. For example, the formulation can be administered as a complementary treatment to antibiotics when a patient is suffering from an acute infection, to reduce the risk of recurrence after an acute infection has subsided, or when a patient will be in close proximity to others with or at risk of serious gastrointestinal infections (physicians, nurses, hospital workers, family members of those who are ill or hospitalized).

The present formulations can be administered to animals, including humans, laboratory animals (e.g., primates, rats, and mice), livestock (e.g., cows, sheep, goats, pigs, turkeys, and chickens), and household pets (e.g., dogs, cats, and rodents). In some embodiments, the formulation is administered to a human subject. In some embodiments, the human subject has one or more signs or symptoms of an IBD (e.g., ulcerative colitis or Crohn's disease), for example, diarrhea (e.g., containing blood or pus); abdominal pain and cramping; rectal pain; rectal bleeding; urgency to defecate; inability to defecate despite urgency; weight loss; fatigue; fever; failure to grow (in children); severe bleeding; perforated colon; severe dehydration; liver disease; osteoporosis; inflammation of the skin, joints, or eyes; mouth sores; increased risk of colon cancer; toxic megacolon; or increased risk of blood clots in veins and arteries. A therapeutically effective treatment using a formulation provided herein can ameliorate one or more of such signs and symptoms.

In some embodiments, the subject (e.g., human patient) receives a pretreatment protocol prior to administration of the formulation, wherein the pretreatment protocol prepares the gastrointestinal tract to receive the bacterial composition. In certain embodiments, the pretreatment protocol comprises an antibiotic treatment, wherein the antibiotic treatment alters the bacteria in the patient. In other embodiments, the pretreatment protocol comprises a colonic cleansing (e.g., enema), wherein the colonic cleansing substantially empties the contents of the patient's colon. As used herein, “substantially emptying the contents of the colon” refers to removal of at least 75%, at least 80%, at least 90%, at least 95%, or about 100% of the contents of the ordinary volume of colon contents. Antibiotic treatment can precede the colon-cleansing protocol.

In some embodiments, a pretreatment protocol is administered to the patient at least 1 day, 2 days, 3 days, 5 days, 6 days, 7 days, 10 days, or 15 days prior to administration of the formulation described herein. In some embodiments, the subject receives multiple doses of a formulation. In some embodiments, the subject has at least one sign or symptom of an IBD (e.g., ulcerative colitis or Crohn's disease) prior to administration of the formulation. In other embodiments, the subject does not exhibit a sign or symptom of an IBD (e.g., ulcerative colitis or Crohn's disease) prior to administration of the formulation, e.g., the formulation is administered prophylactically to reduce the risk of a sign or symptom of active IBD.

In some embodiments, a formulation described herein is administered enterically, in other words, by a route of access to the gastrointestinal tract. This includes oral administration, rectal administration (including enema, suppository, or colonoscopy), by an oral or nasal tube (nasogastric, nasojejunal, oral gastric, or oral jejunal), or any other method known in the art.

In some embodiments, a formulation is administered to at least one region of the gastrointestinal tract, including the mouth, esophagus, stomach, small intestine, large intestine, and rectum. In other embodiments, a formulation is administered to all regions of the gastrointestinal tract. In certain embodiments, a formulation is administered orally in the form of medicaments such as powders, capsules, tablets, gels or liquids. The formulation can also be administered in gel or liquid form by the oral route or through a nasogastric tube, or by the rectal route in a gel or liquid form, by enema or instillation through a colonoscope or by a suppository.

In some embodiments, the bacteria and bacterial compositions are provided in a dosage form. In some embodiments, the dosage form is designed for administration of at least one OTU or combination thereof disclosed herein, wherein the total amount of bacterial composition administered is selected from 0.1 ng to 10 g, 10 ng to 1 g, 100 ng to 0.1 g, 0.1 mg to 500 mg, 1 mg to 100 mg, or from 10-15 mg. In some embodiments, the bacterial composition is consumed at a rate of from 0.1 ng to 10 g a day, 10 ng to 1 g a day, 100 ng to 0.1 g a day, 0.1 mg to 500 mg a day, 1 mg to 100 mg a day, or from 10-15 mg a day, or more.

In some embodiments, the treatment period is at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, or at least 1 year. In some embodiments, the treatment period is from 1 day to 1 week, from 1 week to 4 weeks, from 1 month, to 3 months, from 3 months to 6 months, from 6 months to 1 year, or for over a year.

In some embodiments, from 10⁵ and 10¹² microorganisms total is administered to the patient in a given dosage form. In certain embodiments, an effective amount can be provided in from 1 to 500 ml or from 1 to 500 grams of the bacterial composition having from 10⁷ to 10¹¹ bacteria per ml or per gram, or a capsule, tablet, or suppository having from 1 mg to 1000 mg lyophilized powder having from 10⁷ to 10¹¹ bacteria. In some embodiments, those receiving acute treatment receive higher doses than those who are receiving chronic administration (such as hospital workers or those admitted into long-term care facilities).

In some embodiments, a formulation described herein is administered once, on a single occasion or on multiple occasions, such as once a day for several days or more than once a day on the day of administration (including twice daily, three times daily, or up to five times daily). In some embodiments, a formulation is administered intermittently according to a set schedule, e.g., once weekly, once monthly, or when the patient relapses from the primary illness. In other embodiments, a formulation is administered on a long-term basis to individuals who are at risk for infection with or who can be carriers of these pathogens, including individuals who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

In some embodiments, a bacterial composition of the present disclosure is administered with other agents (e.g., anti-microbial agents or prebiotics) as a combination therapy mode. In certain embodiments, the administration is sequential, over a period of hours or days. In other embodiments, the administration is simultaneous.

In some embodiments, a bacterial composition is included in combination therapy with one or more anti-microbial agents, which include anti-bacterial agents, anti-fungal agents, anti-viral agents and anti-parasitic agents.

Anti-bacterial agents include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem).

Anti-viral agents include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir and Zidovudine.

Examples of antifungal compounds include, but are not limited to polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; and echinocandin antifungals such as anidulafungin, caspofungin, and micafungin. Other compounds that have antifungal properties include, but are not limited to polygodial, benzoic acid, ciclopirox, tolnaftate, undecylenic acid, flucytosine or 5-fluorocytosine, griseofulvin, and haloprogin.

In some embodiments, a bacterial composition is included in combination therapy with one or more corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.

A prebiotic is a selectively fermented ingredient that allows specific changes, both in the composition and/or activity in the gastrointestinal microbiota that confers benefits upon host well-being and health. Prebiotics can include complex carbohydrates, amino acids, peptides, or other essential nutritional components for the survival of the bacterial composition. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

The specification is most thoroughly understood in light of the teachings of the references cited within the specification. The embodiments within the specification provide an illustration of embodiments and should not be construed to limit the scope. The skilled artisan readily recognizes that many other embodiments are encompassed. All publications and patents cited in this disclosure are incorporated by reference in their entirety. To the extent the material incorporated by reference contradicts or is inconsistent with this specification, the specification will supersede any such material. The citation of any references herein is not an admission that such references are prior art.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and can vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

The following examples are offered by way of illustration and not by way of limitation. The contents of all references cited throughout this application are expressly incorporated herein by reference.

### EXAMPLES

### Example 1: Analysis of Bile Acid Levels In\Lcerative Colitis Patients after Administration of a Spore Population Derived from the Feces of Healthy Human (HHSP)

To begin understanding the role of bile acids in inflammatory bowel diseases (IBD), the levels of secondary bile acids associated with the 7α-dehydroxylase pathway (i.e., DCA, LCA, 3-oxo LCA, 3α 12-oxo-deoxycholic acid, and 3β 12α-deoxycholic acid) were quantified in fecal samples obtained from ulcerative colitis patients that received different treatment regimens. The treatment regimens included one of the following: (A) placebo only; (B) placebo followed by a weekly dosing of a spore population derived from the feces of a healthy human (“HHSP”); (C) vancomycin followed by weekly dosing of HHSP; and (D) vancomycin followed by daily dosing of HHSP. Bile acid concentrations were measured at four different time points: (1) baseline (i.e., prior to administration of the vancomycin or HHSP) (“Visit 1”); (2) immediately after vancomycin treatment (where required) (“Visit 4”); (3) 2 weeks after beginning the administration of the HHSP (where required) (“Visit 6”); and (4) 8 weeks after beginning the administration of the spore-forming fraction (where required) (“Visit 12”).

Extraction of Bile Acids from Human Fecal Samples

Human fecal samples were aliquoted, weighed and homogenized in 10× w/v extraction buffer (50% methanol in water). Samples were extracted on ice for 1 hour and then further extracted with an equal volume of cold acetonitrile. Extracts were then centrifuged, and the supernatant filtered through a 0.22 μm filter prior to analysis by liquid chromatography-tandem mass spectrometry (LC-MS). Labeled bile acid standards were spiked pre- and post-extraction to provide quality controls for metabolite extraction and analysis. An aliquot of each sample was also weighed, and then dried to determine sample water content and dry weights.

### LC-MS Analysis of Bile Acids

Bile acids were separated using an Agilent 1260 HPLC equipped with a Microsolv bidentate C18 column preceded by a 0.2 μm pre-column filter. Separation was achieved using a water and acetonitrile gradient with 0.1% formic acid at a flow rate of 0.4 ml/minute. Samples were injected at a volume of 5 μL. The HPLC system was coupled to a Bruker Compass™ qTOF mass spectrometer calibrated to a mass range of 50 to 1700 m/z using the Agilent low-mass tuning mix. Each run was additionally calibrated to a reference mass solution injected at the beginning of each run. Bile acids were detected in negative mode and identified by unique m/z and retention times compared to known pure standards. Area under the peak was determined using Bruker data analysis software. Bile acids were quantified using calibration curves generated from pure standards, ranging in concentration from 0.001 μM to 100 μM. Bile acids detected by LC-MS are listed in Table 1, below.

TABLE 1

| Conjugated 1° | | | Conjugated. |
|---|---|---|---|
| Bile Acids | 1° Bile Acids | 2° Bile Acids | 2° Bile Acids |
| Taurocholic | Cholic acid (CA) | Deoxycholic acid | Tauro-deoxycholic |
| acid (tCA) | Chenodeoxycholic | (DCA) | acid (tDCA) |
| Glycocholic | Acid (CDCA) | Lithocholic acid | Glyco-deoxycholic |
| acid (gCA) | α-Muricholic | (LCA) | acid (gDCA) |
| Tauro- | acid (αMCA) | 7-Oxo-cholic acid | Tauro-lithocholic |
| chenodeoxycholic | β-Muricholic | (7-oxo CA) | acid (tLCA) |
| acid (tCDCA) | Acid (βMCA) | 12-Oxo-cholic acid | Glyco-lithocholic |
| Glyco- | Hyocholic acid | (12-oxo CA) | acid (gLCA) |
| chenodeoxycholic | (HCA) | 3-Oxo-chenodeoxycholic | |
| acid (gCDCA) | | acid (3-oxo CDCA) | |
| Tauro-muricholic | | 7-Oxo-chenodeoxycholic | |
| acid (tMCA) | | acid) 7-oxo CDCA | |
| Tauro-hyocholic | | 12-Oxo-deoxycholic | |
| acid (tHCA) | | acid (12-oxo DCA) | |
| Glyco-hyocholic | | 3-Oxo-lithocholic | |
| acid (gHCA) | | acid) 3-oxo LCA | |
| | | 3β, 12α-deoxycholic | |
| | | acid (Iso-DCA) | |

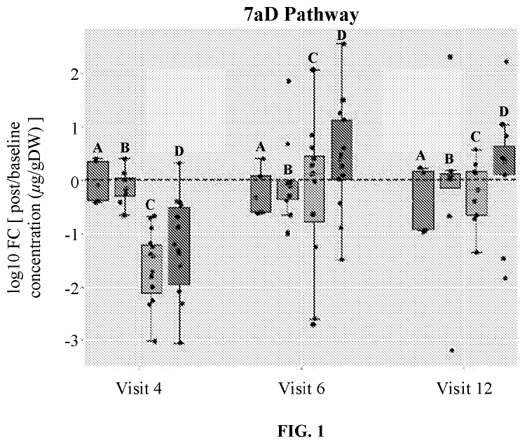

As shown in FIG. 1, ulcerative colitis patients who received vancomycin followed by HHSP (C and D) had increased levels of secondary bile acids associated with the 7α-dehydroxylase pathway in their fecal sample compared to the corresponding levels in the patients prior to the administration. The greatest increase was observed in patients who received vancomycin followed by daily dosing of HHSP (D). In contrast, in patients who received only placebo had no noticeable increase in the concentration of the measured secondary bile acids (A). This result suggests that one or more spore-forming bacteria present in HHSP may be responsible for the increase in bile acid concentrations observed in the ulcerative colitis patients.

### Example 2: Analysis of Bile Acid Levels in Ulcerative Colitis Patients Who are in Remission

To assess the potential relationship between bile acid levels and disease remission, the levels of different bile acids were compared between ulcerative colitis patients who were in remission (“remitters”) and those who had active disease (“non-remitters”). The ulcerative colitis patients had received one of the treatment regimens described in Example 1. The bile acids were extracted from the fecal samples and quantified as described in Example 1, above.

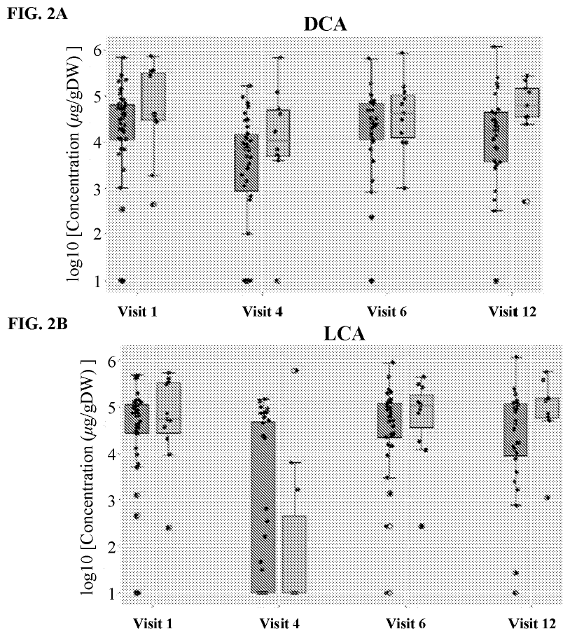

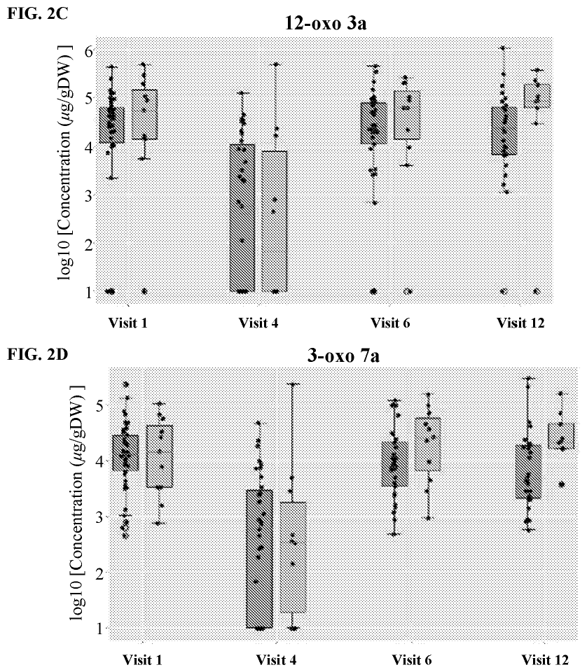

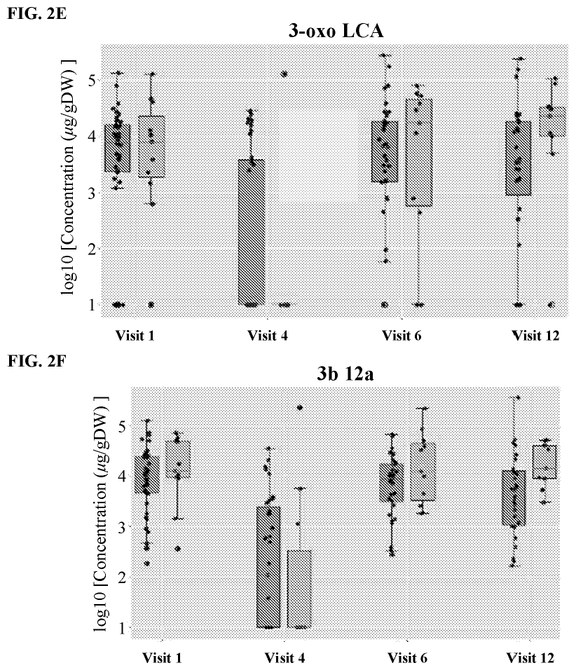

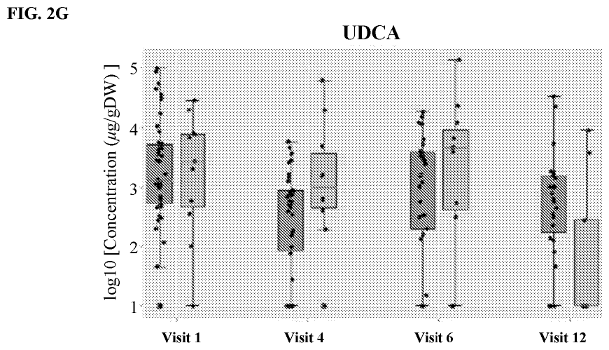

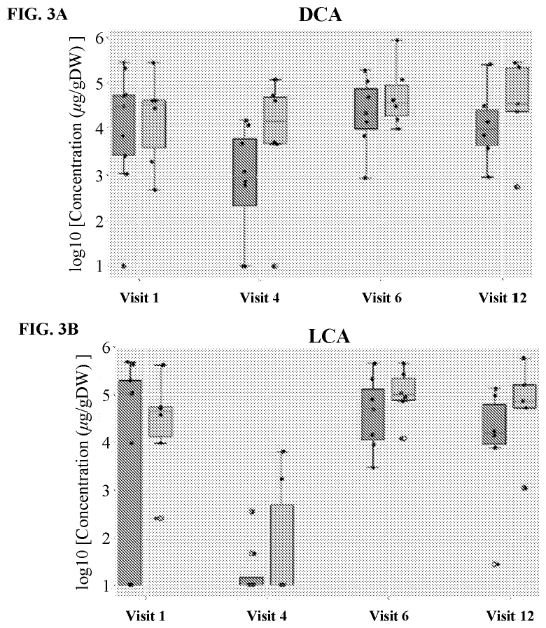

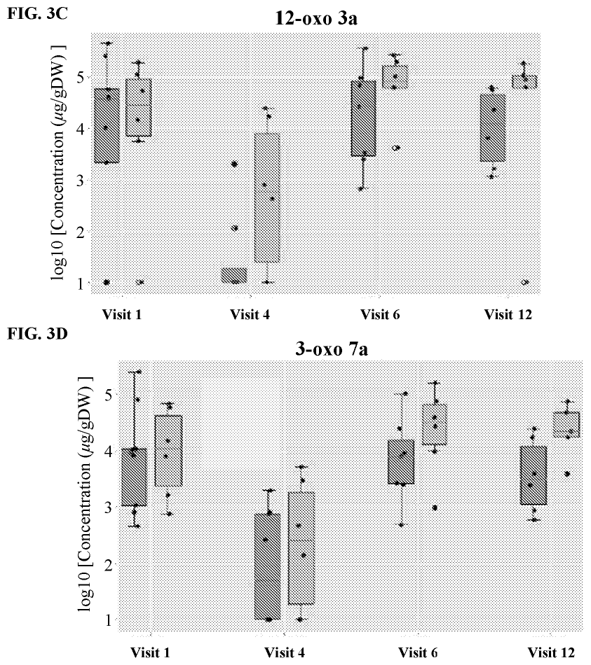

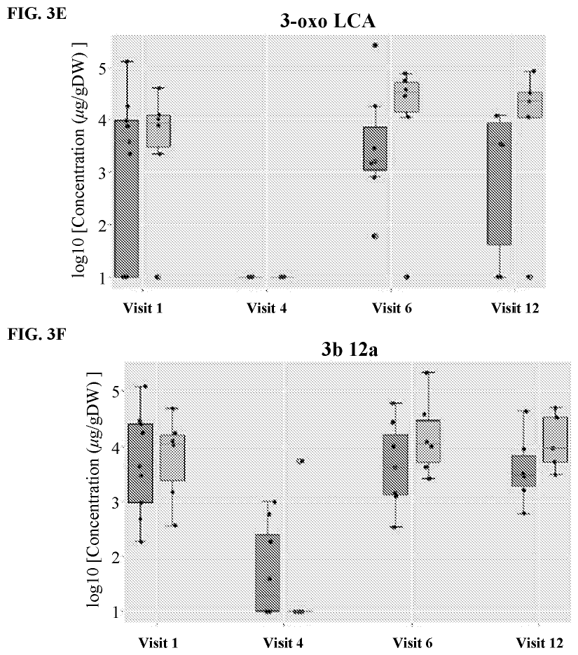

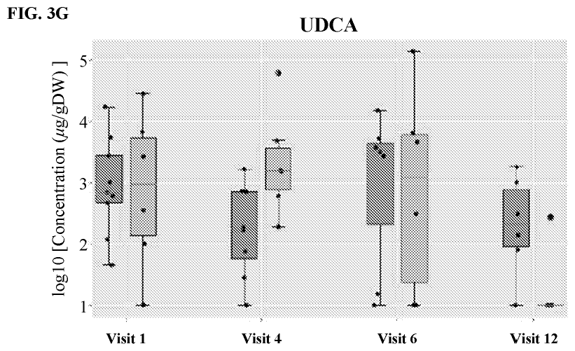

As shown in FIGS. 2A to 2F and FIGS. 3A to 3F, the remitters expressed higher levels of the following secondary bile acids compared to the non-remitters: deoxycholic acid (DCA) (FIGS. 2A and 3A), LCA (FIGS. 2B and 3B), 3α 12-oxo-deoxycholic acid (FIGS. 2C and 3C), 7α 3-oxo-chenodeoxycholic acid (FIGS. 2D and 3D), 3-oxo LCA (FIGS. 2E and 3E), and 3β 12α-oxo-deoxycholic acid (3-isodeoxycholic acid) (FIGS. 2F and 3F). In contrast, the level of UDCA (FIGS. 2G and 3G) was significantly higher in the non-remitters compared to the remitters, suggesting that not all secondary bile acids have similar patterns in inflammatory bowel disease (e.g., ulcerative colitis). The above data further show that IBD remission is directly linked to increase in certain bile acids (e.g., those associated with the 7α-dehydroxylase pathway) and a decrease in levels of UDCA.

These data indicate that manipulation of secondary bile acid levels can be useful for treating an inflammatory bowel disease.

### Example 3: Assessment of Anti-Inflammatory Effects of Secondary Bile Acids on Activated Monocytes and PBMCs

To better understand the possible relationship between the increased level of certain secondary bile acids and ulcerative colitis remission, monocytes and PBMCs were activated with LPS in the presence of varying concentrations of different primary and secondary bile acids in their conjugated and unconjugated forms. The amount of TNF-α and IL-10 produced by the activated cells was measured as described below.

### TNF-α and IL-10 Secretion Assay

Human buffy coat was obtained from Bioreclamation and shipped overnight on ice. Buffy coat was diluted 1:1 with PBS and layered on top of a Ficoll-Paque (GE Healthcare Cat #17-1440-03) in 50 mL falcon tubes. Samples were spun at 500×g for 20 min at room temperature with no brake. PBMCs were aspirated at the Percoll gradient layer and washed 3 times with PBS. Cells were counted with a hemocytometer to determine cell viability and concentration. Cells were plated onto 96 well plate and incubated at 37 C, 5% CO2 for 1 hour. Following this, bile acids were added at a final concentration of 12.5 μM, 25 μM and 50 μM for one hour prior to addition of LPS (1 ng/mL). After incubation of 16-20 hours, the cell culture media were collected for cytokine analysis. Cytokines were assayed using the Milliplex Human Cytokine kit (Luminex; Millipore), assaying IL-10 and TNF-α. Monocytes were isolated from PBMCs using the Miltenyi Biotec Pan Monocyte Isolation Kit (Cat No: 130-096-537) as per manufacturer's instructions and assayed as described above.

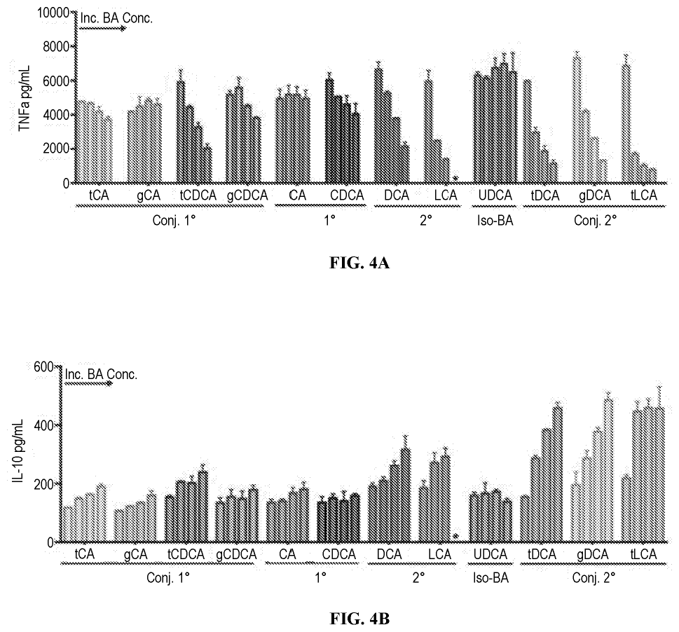

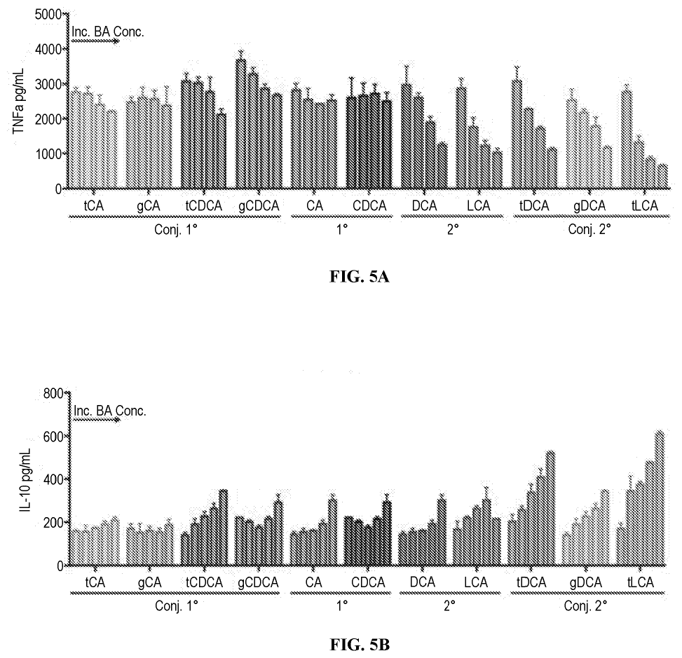

As shown in FIGS. 4A and 4B, with increasing concentration of most of the bile acids shown, the LPS-activated PBMCs produced higher levels of IL-10 and lower levels of TNF-α, highlighting the anti-inflammatory effects of these bile acids. The greatest anti-inflammatory effects were observed with the secondary bile acids (DCA and LCA) and their conjugated derivatives (tDCA, gDCA, and tLCA). Similar results were observed with LPS-stimulated monocytes. See FIGS. 5A and 5B. In agreement with Example 2, increase in UDCA concentration had no anti-inflammatory effects on the LPS-stimulated monocytes or PBMCs.

### Example 4: Assessment of Anti-Inflammatory Effects of Secondary Bile Acids on Activated Intestinal Epithelial Cells

Since IBD primarily affects the intestinal tissues, whether the anti-inflammatory effects observed in monocytes and PBMCs were also true in intestinal epithelial cells (HT29) was assessed as described below.

### IL-8 Secretion Assay

HT29 cells, cultured in McCoys Medium supplemented with 10% FBS, GlutaMAX and Pen/Strep were plated at a density of 50 k cells/well in 96-well format and allowed to grow for 5 days until fully confluent. Culture medium was changed every two days. On day 5, cells were pre-treated for 1 hour with 250 μM, 125 μM or 62.5 μM bile acid compounds before exposure to 1.25 ng/mL TNF-α. Cells were incubated overnight (16 h) and culture supernatants were collected for IL-8 protein quantification by ELISA. IL-8 levels of test samples were normalized to inflammatory controls, DMSO pre-treated samples (i.e., no bile acids) that were exposed to the 1.25 ng/mL TNF-α.

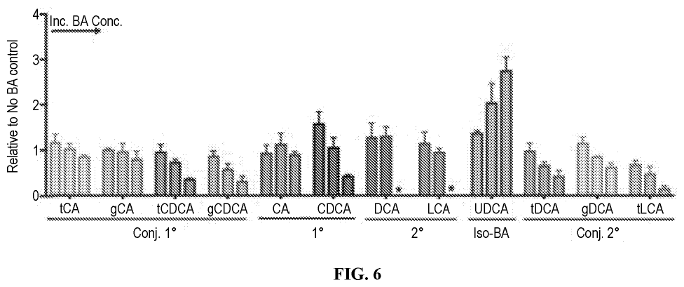

In agreement with the earlier data, intestinal epithelial cells were activated in the presence of several different bile acids, including conjugated secondary bile acids (e.g., t-LCA, t-DCA, and g-DCA) and decreased IL-8 production in a dose-dependent manner. See FIG. 6. Consistent with data provided supra, unlike other tested secondary bile acids, UDCA had no anti-inflammatory effect on the activated intestinal epithelial cells. Instead, UDCA appeared to increase the production of IL-8 by the activated intestinal epithelial cells in a dose-dependent manner.

Collectively, the above data indicates that the increase in certain secondary bile acids (e.g., deoxycholic acid (DCA), lithocholic acid (LCA), or 3-oxo LCA) and/or the decrease in UDCA can enhance anti-inflammatory effects, resulting in disease remission in ulcerative colitis patients.

These data provide further evidence that bacterial compositions that can effect changes in bile acids, e.g., secondary bile acids, can be useful for treating an inflammatory bowel disease.

### Example 5: Identification of Bacteria Responsible for the Increase in Certain Secondary Bile Acids in Remission of Ulcerative Colitis

To identify the bacteria that may be responsible for the increase in bile acids associated with the anti-inflammatory effects described in the above Examples, fecal samples from the different ulcerative colitis patients (i.e., both remitters and non-remitters who received one of the treatment regimens described in Example 1) were divided based on the presence Flavonifractor_SC49 and Clostridium leptum. Then, the amount of secondary bile acids associated with the 7α-dehydroxylase pathway were measured (i.e., DCA, 3α 12-oxo-deoxycholic acid, 3β 12α-deoxycholic acid (3-isodeoxycholic acid), LCA, and 3-oxo-LCA) as described earlier in Example 1.

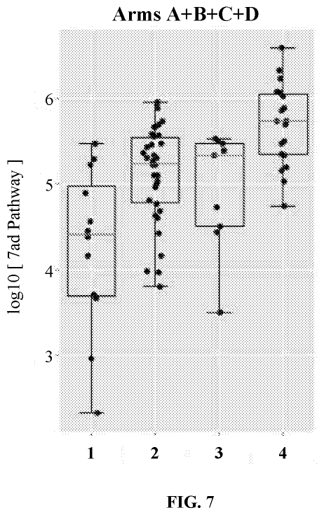

As shown in FIG. 7, fecal samples comprising either Flavonifractor_SC49 or Clostridium leptum had significantly higher levels of the tested secondary bile acids compared to fecal samples that lacked both bacteria. The highest levels of the tested secondary bile acids were measured in fecal samples comprising both Flavonifractor_SC49 and Clostridium leptum. These data indicate that these bacterial species, e.g., in a bacterial composition, can be useful for promoting disease remission in an IBD patient by modulating the levels of certain bile acids in the ulcerative colitis patients.

What is claimed is:

1. A composition comprising a purified population of bacteria, wherein the purified population of bacteria comprises Flavonifractor_SC49, Clostridium leptum, or a combination thereof, and wherein the composition can modulate the level of a secondary bile acid when administered to a subject.

2. The composition of claim 1, wherein the purified population of bacteria comprises Flavonifractor_SC49.

3. The composition of claim 1, wherein the purified population of bacteria comprises Clostridium leptum.

4. The composition of claim 1, wherein the purified population of bacteria comprises both Flavonifractor_SC49 and Clostridium leptum.

5. The composition of any one of claims 1, 2, and 4, wherein the Flavonifractor_SC49 comprises a 16S rDNA sequence that is at least 95%, at least 96%, at least 97%, at least 98%, at least 99%, or 100% identical to a 16S rDNA sequence of a reference Flavonifractor_SC49 OTU (SEQ ID NOs: 1, 3, or 4).

6. The composition of any one of claims 1 and 3 to 5, wherein the Clostridium leptum comprises a 16S rDNA sequence that is at least 95%, at least 96%, at least 97%, at least 98%, at least 99%, or 100% identical to a 16S rDNA sequence of a reference Clostridium leptum OTU (SEQ ID NO: 2).

7. The composition of any one of claims 1 to 6, wherein the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or combinations thereof.

8. The composition of any one of claims 1 to 6, wherein the secondary bile acid comprises ursodeoxycholic acid (UDCA).

9. A method of modulating the level of a secondary bile acid in a subject in need thereof, comprising administering to the subject an effective amount of a composition of any one of claims 1 to 8.

10. A method of ameliorating one or more signs or symptoms of an inflammatory bowel disease (IBD) or maintaining a remission of an IBD in a subject in need thereof, comprising administering to the subject an effective amount of a composition of any one of claims 1 to 8.

11. The method of claim 9 or 10, wherein the secondary bile acid comprises deoxycholic acid (DCA), 3α 12-oxo-deoxycholic acid, 3β 12α-deoxycholic acid (3-isodeoxycholic acid), 7α 3-oxo-chenodeoxycholic acid, lithocholic acid (LCA), 3-oxo LCA, or combinations thereof.

12. The method of claim 11, wherein the administration increases the level of the secondary bile acid in the subject.

13. The method of claim 12, wherein the level of the secondary bile acid is increased by at least 10%, at least 20%, at least 30%, at least 40%, at least 50%, at least 60%, at least 70%, at least 80%, or at least 90% in the subject compared to a reference (e.g., corresponding level in a subject that did not receive the composition).

14. The method of claim 12 or 13, wherein the increase in the level of the secondary bile acid is associated with a remission of the IBD.

15. The method of any one of claims 11 to 14, wherein the secondary bile acid can decrease production of TNF-α and/or increase production of IL-10 in a lipopolysaccharide (LPS)-stimulated monocyte in vitro.

16. The method of any one of claims 11 to 15, wherein the secondary bile acid can decrease production of TNF-α and/or increase production of IL-10 in LPS-stimulated peripheral blood mononuclear cells (PBMCs) in vitro.

17. The method of any one of claims 11 to 16, wherein the secondary bile acid can decrease production of IL-8 in TNFα-stimulated intestinal epithelial cells in vitro.

18. The method of claim 9 or 10, wherein the secondary bile acid comprises ursodeoxycholic acid (UDCA).

19. The method of claim 18, wherein the administration decreases the level of UDCA in the subject.

20. The method of claim 19, wherein the level of UDCA is decreased by at least 10%, at least 20%, at least 30%, at least 40%, at least 50%, at least 60%, at least 70%, at least 80%, or at least 90% in the subject compared to a reference (e.g., corresponding level in a subject that did not receive the composition).

21. The method of claim 19 or 20, wherein the decrease in the level of UDCA is associated with a remission of the IBD.

22. The method of any one of claims 10 to 21, wherein the IBD is ulcerative colitis or Crohn's disease.
